(19) United States
(12) Patent Application Publication
(10) Pub. No.: US 2022/0370316 A1
(43) Pub. Date: Nov. 24, 2022

(54) BIOCIDE PRODUCT OF BROAD, CONTINUOUS AND PROLONGUED EFFECT BASED ON ETHANOL, BENZALKONIUM CHLORIDE AND COPPER NANOPARTICLES

(71) Applicant: MARIA EUGENIA LORENZETTI, BUENOS AIRES (AR)

(72) Inventor: MARIA EUGENIA LORENZETTI, BUENOS AIRES (AR)

(21) Appl. No.: 17/367,554

(22) Filed: Jul. 5, 2021

(30) Foreign Application Priority Data
May 18, 2021 (AR) 20210101360

(51) Int. Cl. A61K 8/34; A61K 8/04; A61Q 17/00; A61K 8/41; A61K 8/19; A01N 31/02; A01N 33/12; A01N 59/20

(52) U.S. Cl. CPC A61K 8/34; A61K 8/042; A61Q 17/005; A61K 8/416; A61K 8/19; A01N 31/02; A01N 33/12; A01N 59/20

(57) ABSTRACT

A biocide product comprising biocidal agents, surfactants and solvents characterized in that it comprehends the following elements: a) Alcohol taken from the following group: ethyl alcohol, methyl alcohol and isopropyl alcohol; b) Quaternary ammonium taken from the following group: Benzalkonium chloride, Ethyl Benzyl Chloride, dodecyl dimethyl ammonium chloride and the combinations thereof. The different formulas that may be prepared, in liquid as in gel, all of them contain alcohol, quaternary ammonium and copper nanoparticles in a determined range of proportions. The use of the obtained biocide in the liquid formula results more apt to be applied on surfaces and the gel formulation for personal use especially on the skin.

### FIELD OF THE DISCLOSURE

The present invention is about a biocide product, of continuous and prolonged effect that comprises alcohol as a biocidal agent of immediate action, copper nanoparticles as biocidal agent of prolonged effect and a quaternary ammonium, particularly benzalkonium chloride, as biocide of intermediate action.

### BACKGROUND OF THE DISCLOSURE

### State of the Art

The problems involving disinfection and sanitization have been and are still a great concern in the health care field, mainly in the hospital environment in relation to the healthcare associated infections (HAIs) and have increased in importance associated to disinfection at homes as well as commercial, industrial and transportation facilities, and mainly related to different epidemics of the last decades, and lately in particular to COVID-19 pandemic.

Within several used biocide products, we are going to particularly mention alcohol, quaternary ammoniums and copper nanoparticle.

Ethylic and isopropyl alcohols are water soluble organic compounds, historically employed in medicine as cleaning antiseptics and disinfection of injuries. Alcohols supply quick effect and of ample spectrum. In addition to the antimicrobial activity, alcohols are effective solvents of other products, as well as many antiseptics and disinfectants that maximize such activity. Alcohols usually employed are ethylic alcohol (ethanol) and isopropyl alcohol. Concentrations may vary between 70 and 96% for the former and 70 and 100% for the latter. Said alcohols destroy the cell membrane by reduction of its superficial stress, and by denaturalizing proteins. The efficiency is based in the presence of water, thus, said alcohols penetrate in a better manner in cells and bacteria allowing the damage to the membrane and a quick denaturalization of the proteins, with the consequential interference with the cellular lysis and metabolism. The action of said alcohols is quick, even as from 15 seconds, mainly in concentrations of 70%, said concentration allowing an enhanced penetration in the bacterial protoplasm. The biological effects of microbial damage of said alcohols are mostly brief. Ethanol, at 70% concentration, destroys approximately 90% of the cutaneous bacteria in two minutes, provided that the skin remains in contact with said alcohol without letting it dry. Said alcohols are inactivated with the presence of organic matter.

Furthermore, an alcohol solution in a concentration of 70%, applied on domestic surfaces will lose effect after 3 or 4 minutes due to the evaporation of alcohol and the resulting decrease of the biocidal capacity, said time interval depending on several conditions, among said conditions we can mention, pressure and temperature of the environment. Moreover, investigations carried out in Brazil confirm that the efficiency of alcohol applied directly on contaminated surfaces does not vary if said surface has been previously cleaned (Graziano M U Graziano K U, Pinto F M G, Bruna C Q M, Queiroz R Q, Lascala C A. Efficiency of disinfection with alcohol at 7% (p/v) on contaminated surfaces with no previous cleaning. Rev. Latino-Am Enfermagem [Internet]. March-April 2013).

The quaternary ammoniums correspond to a family of compounds, their basic structure being ammonium cation (NH4+) and, when being modified results in different disinfectant agents. Said quaternary ammoniums are soluble in water and alcohol, function in acid and neutral means, being more efficient in alkaline means depending of their chemical nature. Said quaternary ammoniums having surfactant properties and the activity being decreased with the presence of organic matter. Said quaternary ammoniums present a disinfectant action in concentrations as from 0.05% or higher, mainly used on surfaces of clinical furniture and physical plants in hospital facilities. For said purposes said ammoniums are used in aqeuous solutions or mixed with detergents to apply in one motion combining cleanliness and disinfection. Said quaternary ammoniums are generally associated to tertiary amines in disinfectant formulas resulting in an increase of said biocide action. Quaternary ammonium salts are generally recognised as colourless or yellow compounds, odourless, deodorant and non-irritant in concentrations of habitual use.

Benzalkonium chloride is a cationic cleanser of quaternary ammonium that is included as part of the non-oxidant biocide group. Benzalkonium chloride was the first compound of quaternary ammonium used as an antiseptic and maintains a wide use in disinfection in hospitals until nowadays; said molecules presented an alkyl group with most quantity of carbons sustaining a greater antimicrobial power. The antimicrobial range is very broad including bacteria (gram-positive), fungi (Epidermophyton, Trichophyton and Candida albicans) and protozoans (Entamoeba histolytica and Trichomona vaginalis). In general they are active before a virus with lipid coating. Its action being higher than a neutral PH or lightly alkaline, as well as in the presence of alcohols, on the other hand it is lower that an acid PH and in the presence of organic matter. After the topical application, the result is immediate and the time range of effectivity may last until a maximum of 12 hours.

The most used and studied metallic nanoparticles concerning its antibacterial effect are titanium dioxide, silver and zinc oxide. However, most recently copper nanoparticles (Cu NPS) has had a focused interest due to their antimicrobial and antifungal properties. Its effectivity is evidenced before bacteria such as Escherichia coli (3-5), Pseudomona aeruginosa (6), Klebsiella pneumoniae (5), Enterococus faecalis, Shigella flexneri, Salmonella typhimurium (7), Proteus vulgaris and Staphylococcus aureus. Also it has been noticed that CuO nanoparticles, when exposed to bacteria such as Micrococcus luteus, Staphylococcus aureus, Escherichia coli, Klebsiella pneumoniae and Pseudomonas aeruginosa, show a higher inhibitory activity compared to the halo of inhibition provided by fungi such as Aspergillus flavus, Aspergillus niger and Candida albicans. Other investigations confirm the inhibitory effect of CuO nanoparticles before bacteria Gram Positive and Gram negative, where the size of the nanoparticles is correlated with its antimicrobial activity.

The methods to obtain the nanoparticles formed as from copper oxo-hydroxide capable of supplying biocide concentrations of copper, normally in the form of free copper ions (Cu²⁺) have been described in several publications, among such, patent WO2016/170152 by Medical Research Council (GB) or the method of precipitation developed by Rios Valer (Rios Vales, G. B Morphological and structural study of CuO nanoparticles employing dispersion techniques of light and X rays Bach. Thesis, National Engineering University, Peru, 2015). Furthermore, other patents reveal biocide products with copper nanoparticles and the method for obtaining it, as patent MX2017013704 (New method for obtaining solver-copper bimetallic nanoparticles as from de mixture of copper compounds), or patent CL2017001504 (Chemical synthesis of copper sulphide nanoparticles), or patent CL2015003652 (Translucent adhesive film with antibacterial activity, for the protection of surfaces, comprising copper nanoparticles, method for preparation and use for the protection of surfaces with high exposure to the presence of bacteria and fungi), or patent CL2015000856 (Procedure fort the elaboration and obtaining of a concentrated synergic base mixture of colloidal nanoparticles electrolytically merging, at least two different, high purity or innocuous copper, silver, gold platinum or zinc bars or rods, for the elaboration of biocide products), or patent MX2012012726 (Process for the synthesis of copper nanoparticles by means of the thermal decomposition and its use as antimicrobial agents).

With the support of the accumulated scientific evidence, on Mar. 25, 2008, the EPA (Environmental Protection Agency), registered copper as the first and only metal with antibacterial properties, authorizing the diffusion of important concepts, such as that “the copper surfaces eliminate 99.9% of the bacterial pathogens after 2 hours of being exposed” and certifying that metallic copper surfaces and their alloys are natural antimicrobials, possess a long term antimicrobial efficiency, have an autonomous disinfectant effect and are superior to other coatings available in the market. This registry authorizes the use of copper surfaces in health care facilities. Laboratory evidences register the efficiency of copper to eliminate spores and vegetative forms do Clostridium difficile, hospital pathogen associated to outbreaks of IIH with a high level of mortality. These studies showed the elimination of spores after 24 hours of exposure to metallic copper, and another study shows that this effect occurs as from 30 minutes for the vegetative forms and as from 3 hours for the spores, even before the presence of organic matter.

Fundamentally a good biocide must have a wide range of activity, that is, it must cover a wide range of microorganisms (bacteria, virus and fungi) and, the lower the concentration the more economic will be the resulting product. It is also advisable that it is effective in a wide range of pH, that it is soluble in water and alcohol, and that it is compatible with other chemical species in the chemical environment. Last, it is also desirable that it has a high persistency, and, to be easy to neutralize and its toxicity to be very low or inexistent.

The state of the art teaches that alcohol is a biocide of immediate action, but it presents a high volatility, consequently with a fast loss of its effective concentration. It also teaches that the action of copper nanoparticles start its biocidal action after 30 minutes or more depending on the kind or organism, but its biocide action can remain for several days when applied on a surface or when it is part of laminations for surfaces. Moreover, the quaternary ammoniums start their action quickly, but, as they do not have the high level of volatility of alcohol, and also that it adheres to surfaces, its biocidal effect may last several hours, but decrease their action in acid means and over time.

Such as it is shown from the state of the art, one of the drawbacks of the copper nanoparticles is that they tend to oxidize easily, as, due to its low redox potential results in the formation of a layer of rust in the surface of the nanoparticles, thus producing the modification of its antimicrobial effect. Another drawback is the agglomeration of the nanoparticles its effectivity increasing when being dispersed.

### SUMMARY OF THE DISCLOSURE

The present invention forwards a solution to the problem of continuity of the biocidal action by combining alcohol, benzalkonium chloride and copper nanoparticles, maintaining a neutral or alkaline environment to the effects of not diminishing the effectivity of the components. Moreover, the coordinated action in the time of the combination of alcohol, benzalkonium chloride and copper nanoparticles provide an improved effectivity and boosts the individual action of each one of the biocides preventing the accumulation of viruses, bacteria and fungi, enhancing the action of these other biocides, and particularly, the copper nanoparticles are affective on microorganisms.

### DETAILED DESCRIPTION OF THE DISCLOSURE

The present invention is about a biocide product, of continuous and prolonged effect that comprises alcohol as a biocidal agent of immediate action, copper nanoparticles as biocidal agent of prolonged effect and a quaternary ammonium, particularly benzalkonium chloride, as biocide of intermediate action.

The different formulas that may be prepared, in liquid as in gel, all of them contain alcohol, quaternary ammonium and copper nanoparticles in a determined range of proportions.

A preferred aspect of the present invention is that the final biocidal product to contain a proportion of alcohol between 65% and 75%, quaternary ammonium between 0.05% and 2.25% and copper nanoparticles between 5 to 100 ppm (parts per million).

It is also another preferred aspect that the alcohol used as biocidal to be ethanol, and that the quaternary ammonium to be Benzalkonium Chloride (Alkyl-benzyl-dimethyl ammonium chloride).

One of the formulas of the present invention, —biocide gel of prolonged effect—is embodied in the following manner: 23.85 ml of demineralized water at 40° to 50° centigrade is poured in a tank, that we shall name main tank. A dispersion of nanoparticles in water, with 0.001 gr. of copper nanoparticles in demineralized water is prepared in an auxiliary tank, and it is stirred for at least 2 minute at 3000 rpm.

The stirrer of the main tank is started, acting at 1400 rpm and the mentioned previously prepared dispersion of nanoparticles is poured without stopping the stirrer. Immediately, the gelling powder is added in a quantity of 1.1 gr, in a slow motion and continuous stirring so as to prevent the formation of lumps. The stirring is continued in the main tank for 10 to 15 minutes until a homogeneous and transparent gel is obtained. Without interrupting the stirring 0.06 ml. of benzalkonium chloride in the form of a solution at 80% is added. Then ethanol 65 ml is added on the main container and stirred until the total content of the batch (100 ml) is completed, stirring is continued for at least 15 minutes more. Following exactly said steps and the proportions of the components the manufacturing of the biocide gel product of prolonged duration is obtained.

Thus a preferred formulation that can be applied on the skin comprises, for 100 ml of the final product: Ethanol 70% (70 ml), Benzalkonium Chloride 0.05% (0.05 ml); copper nanoparticles 10 ppm (0.0001 gr), demineralized water 29.95% (29.95 ml).

Another embodiment of the present invention comprises a liquid formulation where the same steps as aforementioned are repeated but without the use of the gelling agent.

The use of the biocide in the liquid formula results more apt to be applied on surfaces and the gel formulation for personal use especially on the skin.

The following formulation is preferred to be used on surfaces, furniture and facilities for 100 ml of the final product: Ethanol 60% (60 ml); Benzalkonium Chloride 0.25% (0.25 ml); Copper nanoparticles 10 ppm (0.0001 gr), demineralized water 39.75% (39.75 ml).

Such as it has been described, the invention has multiple uses not only personal use for hands and skin but also on surfaces for support and contact for domestic use, countertops and furniture for commercial and industrial facilities, interior of means of transport (private or public), healthcare facilities, schools, etc., due to its wide range and prolonged duration of its biocidal effect.

1. A biocide product with biocidal agents, surfactants and solvents comprising the following biocidal agents:
a) Alcohol taken from the following group: ethyl alcohol, methyl alcohol and isopropyl alcohol.
b) Quaternary ammonium taken from the following group: Benzalkonium chloride, Ethyl Benzyl Chloride, dodecyl dimethyl ammonium chloride and the combinations thereof.
c) Metallic nanoparticles taken from the following group: titanium dioxide, silver, copper and zinc oxide and the combinations thereof.

2. The biocide product in accordance to claim 1 comprising that the proportion of the biocide agents are:
Alcohol in a volume between 60% and 75%
Quaternary ammonium in a volume between 0.05% and 2.25%.
Metallic nanoparticles between 5 and 100 ppm (parts per million).

3. The biocide product in accordance to claim 2 comprising that the product includes the following biocide agents in the indicated proportions:
Ethanol 70% (70 ml); Benzalkonium Chloride 0.05% (0.05 ml); Copper nanoparticles 10 ppm (0.001 gr).

4. The product in accordance to claim 2 comprising in that the product includes the following biocide agents in the indicated proportions:
Ethanol 60% (60 ml); Benzalkonium Chloride 0.25% (0.25 ml); Copper nanoparticles 10 ppm (0.001 gr).

5. The product in accordance to claim 3 comprising in that a gelling mater is added in order to improve the application of said product on skin and hands.
